(19) United States
(12) Patent Application Publication
(10) Pub. No.: US 2019/0114719 A1
(43) Pub. Date: Apr. 18, 2019

(54) Dynamic balance adjustment estimator engine

(71) Applicant: Thomas T. Brekka, Santa Cruz, CA (US)

(72) Inventor: Thomas T. Brekka, Santa Cruz, CA (US)

(73) Assignee: VestaCare, Inc., La Jolla, CA (US)

(21) Appl. No.: 16/350,547

(22) Filed: Dec. 1, 2018

Related U.S. Application Data
(63) Continuation-in-part of application No. 15/731,777, filed on Jul. 31, 2017.

(51) Int. Cl. G06Q 40/08; G16H 30/20; G06F 16/81

(52) U.S. Cl. CPC G06Q 40/08; G06F 16/81; G16H 30/20

(57) ABSTRACT

A dynamic estimator engine for obtaining patient information, encounter information and insurance information from a hospital, clinic or other provider system, for performing calculations to compute reliable patient payment estimates at or prior to the time of service which enables hospitals or medical providers to increase collections and decrease patient bad-dept. The method is implemented through the operation of a computer, or a central data processing unit, and may utilize using wireless communication channels, storage media, computer buses, or other data processing and transfer mechanisms.

### CROSS REFERENCE TO RELATED APPLICATIONS

This Patent Application is a continuation-in-part of and claims priority from U.S. patent application Ser. No. 15/731,777 filed Jul. 31, 2017.

### BACKGROUND OF THE INVENTION

### Technical Field

This invention relates to estimator engines and methods for performing dynamic balance adjustments to compute reliable patient payment estimates at or prior to the time of service which enables hospitals or medical providers to increase collections and decrease patient bad-dept.

### Background Art

Heretofore, numerous accounting systems and methods have been applied and used by hospitals and medical providers. Various methods have been proposed and implemented for hospital and medical provider's billing and accounting procedures.

Although prior methods have been adapted and used, applicant is not aware of any method specifically for performing dynamic balance adjustments to compute reliable patient payment estimates at or prior to the time of service which enables hospitals or medical providers to increase collections and decrease patient bad-dept.

In the current healthcare system, patients have increasing financial responsibilities for their healthcare services, and accordingly it is important for the provider through the use of an estimation method to provide to the patient probable projections of financial responsibilities and liabilities. The present methodology provides a novel estimator engine to effectively communicate to patients about the expected financial responsibilities at or prior the time of service.

A crucial component of the present estimator engine is insurance eligibility verification. The ability to verify the patient coverage including copays, coinsurance and deductible information via integration with third-party eligibility services and augmenting any missing information by scanning the individual payer websites through the use of robotics automations enable providers to capture accurate patient eligibility and benefit information.

The present methodology provides, for the first time, a method and system capable of generating reliable patient payment estimates via an estimator engine that can seamlessly integrate with any existing estimator system being utilized by the provider. It further allows user staff to compare estimates between different systems and choose the appropriate one.

The estimator engine of the present invention has the flexibility to adapt to multiple situations in which an estimate is needed for pre-registered services. These situations include: patients existing in the system and “on-demand” patients calling in with pricing questions. In application, when using the present estimator engine, patients get the transparency they need about payment information while allowing the provider staff to collect payments or enroll patients on automatic payment plans upfront.

Accordingly, the primary object of the present invention is to provide a method and system for performing dynamic balance operations using an estimator engine, for performing calculations to compute reliable patient payment estimates at or prior to the time of service which enables hospitals or medical providers to increase collections and decrease patient bad-dept.

Additional objects and advantages of the invention will be set forth in the description which follows, and in part will be obvious from the description, or may be learned by practice of the invention. The objects and advantages of the invention may be realized and obtained by means of the methods, instrumentalities, and combinations particularly pointed out in the appended claims.

### SUMMARY OF INVENTION

To achieve the foregoing objects, and in accordance with the purpose of the invention as embodied and broadly described herein, an estimator engine is provided, for obtaining patient information, encounter information and insurance information from a client system, and for performing calculations to compute reliable patient payment estimates at or prior to the time of service which enables hospitals or medical providers to increase collections and decrease patient bad-dept. The method is implemented through the operation of a computer, or a central data processing unit, and may utilize using wireless communication channels, storage media, computer buses, or other data processing and transfer mechanisms. The present method utilizes software, cloud computing, internet and other digital operations in application.

### BRIEF DESCRIPTION OF DRAWINGS

The accompanying drawings, which are incorporated in and constitute a part of the specification, illustrate a preferred embodiment of the invention and, together with a general description given above and the detailed description of the preferred embodiment given below, serve to explain the principles of the invention.

FIG. 1 is a schematic diagram of a preferred method of or receiving HL7 ADT messages from a hospital system to synchronize patient demographics, according to the invention.

FIG. 2 shows a preferred architecture of the estimator engine, according to the invention.

FIG. 3 is a schematic diagram of a preferred method of a patient's responsibility calculation sequence, according to the invention.

FIG. 4 is an illustration of a typical health insurance plan cost sharing between insured and insurer, and shown herein to illustrate an application of the present invention.

FIG. 5 shows an eligibility data view, according to the invention.

FIG. 6 shows an out of pocket maximum remaining data view, according to the invention.

FIG. 7 shows a patient pays deductible and co-insurance data view, according to the invention.

FIG. 8 shows an example of a patient pays deductible only data view, according to the invention.

FIG. 9 shows an example of a patient pays coinsurance only data view, according to the invention.

FIG. 10 shows an example of a patient pays Out of Pocket (OOP) data view, according to the invention.

FIG. 11 shows an example of when information is not sufficient to calculate patient responsibility, according the the invention.

FIG. 12 shows an example of a situation where a primary insurance deductible was met and secondary information is present data view, according to the invention.

FIG. 13 is a schematic diagram of various business checks used in order to determine the patient payment responsibility, according to the invention

FIG. 14 shows a method of integrating with existing estimator systems to retrieve ready-to-use estimates and to compare with estimates generated by the present estimator engine, according to the invention

### DETAILED DESCRIPTION OF THE PREFERRED EMBODIMENT

Reference will now be made in detail to the present preferred embodiments of the invention as illustrated in the accompanying drawings.

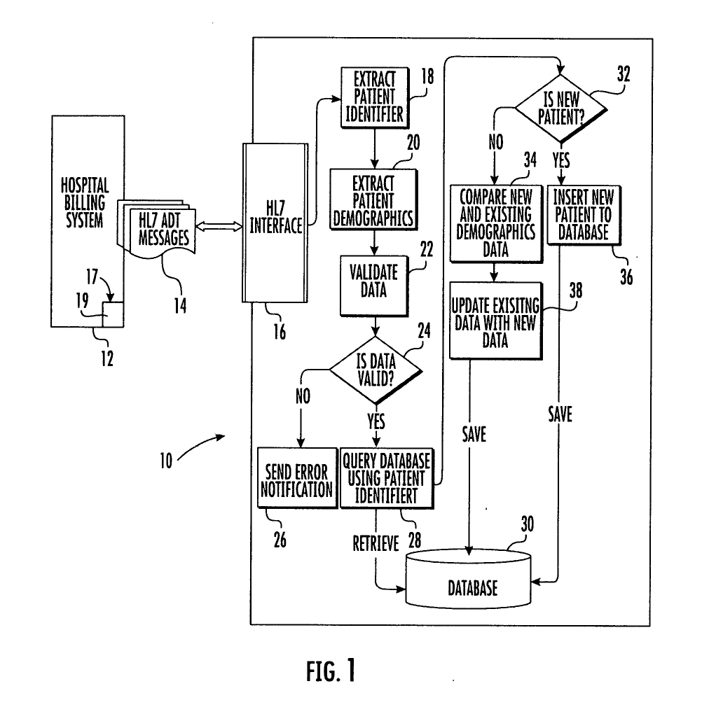

In accordance with the present invention, and as seen in FIG. 1, there is provided in a preferred embodiment of the invention, a dynamic balance adjustment estimator engine 10, methodology for obtaining patient information, encounter information and insurance information from a client system 12, such as a hospital, medical center, clinic or the like. The client system will have computer(s) 19, or other digital processing means, such as tablets, mobile devices, with display screen(s) 17, or other display mechanisms well known in the art.

As seen in FIG. 1, the estimator engine methodology receives patient demographics and visits information via Admission, Discharge, Transfer (ADT) messages 14, in Health Level 7 Protocol (HL7) from the hospital system. These messages contain information such as: patient has been admitted, discharged, transferred, merged, demographics data has changed (name, insurance, address, etc.) or visit information has changed (location, etc.).

The estimator engine 10, interfaces and reads 16, the incoming HL7 ADT messages, extracts the needed information from them and uses that information to locate the corresponding patient records in estimator engine 10, and update them accordingly.

A preferred methodology is seen in FIG. 1, including the steps: extract patient identifier 18, extract patient demographics 20, validate data 24, evaluate if data is valid 24, if no, send error notification 26, if yes, query database 30, using patient identifier 28. Is this a new patient 32, if no, compare new and existing demographics data 34, if yes, insert new patient to database 36. If new data, then update existing data with new data 38, and save to database 30.

In the situation where the client system 14, such as a hospital system can not afford to provide full HL7 capability, the present method allows receiving data through batch files via File Transfer Protocol (FTP). Comma Separated Values (CSV), Excel and other file types are supported. Estimator engine 10, is able to run several channels in parallel to handle the load and process incoming data whether from HL7 or batch files.

In addition, the present methodology may utilize an Automated Posting Application (APA) robotic engine for data extraction by interacting directly with the host hospital billing system. Preferably, an APA robotic engine is used whenever some data cannot be exported into files from the hospital system for some reason or the export operation cannot be automated. APA uses native text extraction methods or Optical Character Recognition (OCR) methods to read the data directly from the hospital system and send it via a secure channel to estimator engine 10, for reconciliation and storage. Using any combination of these methods, the present methodology integrates seamlessly with the hospital, medical center, clinic or the like billing system and receives any updates related to patient demographics, appointments, visits, and the like.

Estimator engine 10, of the present invention also provides a method of retrieving advanced eligibility information by integrating with third-party eligibility services and augmenting missing information from individual payer websites using robotics automation.

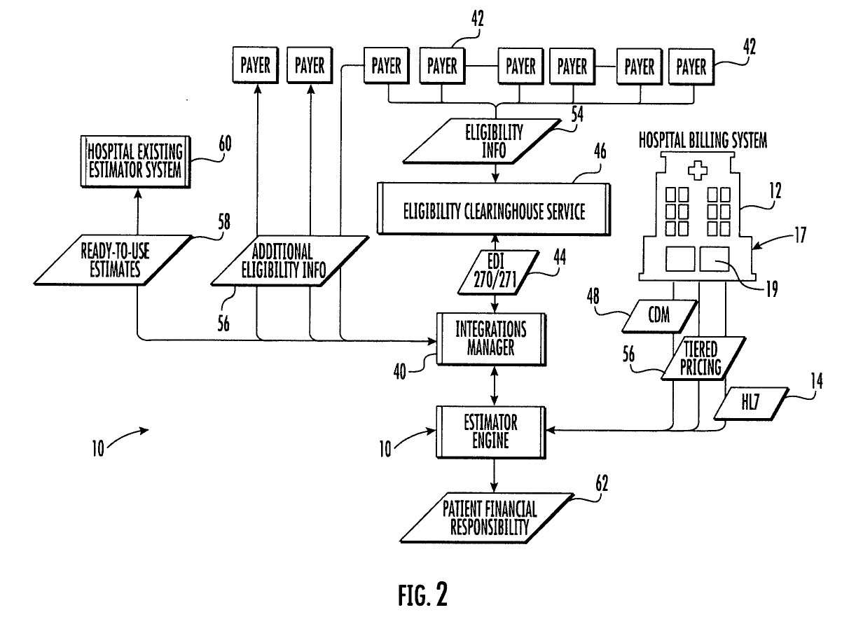

In a preferred embodiment, estimator engine 10, connects through integrations manager 40, with a web service, such as Extensible Markup Language (XML)/Simple Object Access Protocol (SOAP)) with an Eligibility Clearinghouse Service, 46, seen in FIG. 2, to submit and retrieve healthcare transactions. Clearinghouse services provide synchronous and asynchronous communication methods and both are supported in the present methodology. It is preferred to use synchronous method because it provides almost real-time eligibility information retrieval.

Preferably, the web service is SOAP 1.1 and 1.2 compliant. Security is preferably handled through the use of a SOAP header that contains the login credentials. The Eligibility Clearinghouse Service, 46, provides estimator engine 10, with a 99.9+% clearinghouse uptime to ensure minimal disruption of our workflow and with connectivity to more than 800 payers covering 98% of United States insured population lives. In estimator engine 10, patient coverage is verified, including copays, coinsurance, deductible and out-of-pocket information upfront through eligibility requests/responses using the EDI 270/271 standard, seen in FIG. 2, as 44.

In general, even if accurate information is provided with an efficient and secure exchange for healthcare transactions, there are instances where sometimes the provided information can be insufficient for the estimator engine 10. For example, there might be pieces of information that the payers are not returning to the clearinghouse service through any of their multiple connectivity endpoints and this is when the present methodology has the capability of building its own interfaces to the payers' systems.

Integrations manager 40, is capable of interacting directly with payer's websites in order to retrieve the needed additional eligibility information. Its flexibility allows mapping the model of any payer website. When it runs, it navigates through the website pages, captures data, clicks hyperlinks, fills out forms and other functions a regular user would do in a normal browser. This helps in capturing any missing eligibility information that could not be retrieved through the clearinghouse service's EDI 270/271.

As illustrated in FIG. 2, the client system 12, here a hospital billing system, will have computer(s) 19, or other digital processing means, such as tablets, mobile devices, with display screen(s) 17, or other display mechanisms well known in the art. Hospital billing system 12, through Charges Description Master (CDM) 48, tiered pricing 56, and HL7 messages 14, feed information into estimator engine 10. Payer 42, provides eligibility information 54, to Eligibility Clearing house 46, and via DEI270/271, 44, to integrations manager 40. The hospital's existing estimator system 60, is provided by ready to use estimates 58, from integrations manager 40, and may provide prior estimations to the integrations manager 40, which is communicatively linked to estimator engine 10, which thereby provides accurate and detailed patient financial responsibility 62.

The preferred embodiment of estimator engine 10, allows healthcare providers to determine an estimated patient payment responsibility. By using reliable information as input, combining it with the provider's Charges Description Master (CDM) 48, and a set of rules, an estimated patient payment responsibility can be produced that can be used for enrolling patients on an automatic payment plan.

At the core of the revenue cycle, a hospital CDM is extensive. It contains thousands of individual charges and procedures across all hospital departments, usually up to 45,000 or more separate line items. Each charge code is then associated with a revenue code linking to revenue categories used in the hospital's accounting and billing systems. Every chargeable item in the hospital must be part of the CDM in order for a hospital to track and bill a patient, payer, or another healthcare provider. This includes all services and supplies for all patient types.

In the present methodology, all revenue codes have been mapped to one or more of the 187 X12 service type codes available to be submitted in an EDI 270, Loop ID 2110C, EQ segment, element EQ01.

These codes identify business groupings for healthcare services or benefits and they allow us to receive specific data related to a patient's service.

Each CDM record contains an allowed amount and a contracted rate for each one of the payers the hospital has agreement with. Also, as mentioned before, each CDM is associated to a single revenue code and each revenue code has one or multiple service codes assigned to it.

Preferably, each time a new CDM 48, is selected from the Patient Estimates section of the estimator engine 10, solution by a user, new eligibility requests are submitted to the eligibility clearinghouse service with the service codes associated to the revenue-code/CDM relationship and the responses are consolidated into a single and user friendly view on the estimator engine 10, portal. Using this view, the user is able to find out the copay associated to the selected CDM record(s).

Using the present methodology, the user 64, is able to visualize the primary and secondary's deductible, deductible remaining, out of pocket and out of pocket remaining for the individual and family categories associated to the American National Standards Institute (ANSI) service type code “Health Benefit Plan Coverage”. All the previously mentioned information is used to calculate the primary and secondary Deductible, Co-Insurance and Deductible, which is totalized under the Patient Responsibility field, which once confirmed, is assigned to a specific encounter in the present method.

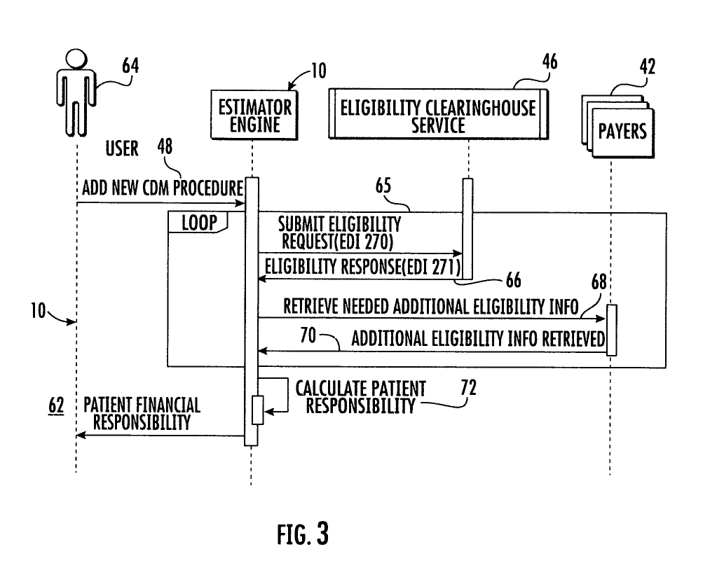

The dynamic balance adjustment estimator engine 10, of the preferred methodology uses eligibility information, CDM, and Tiered Pricing to compute the patient financial responsibility 62. Estimator engine 10, preferably uses the following information when calculating the patient payment responsibility as seen in FIG. 3. Eligibility information, comprising submit eligibility request 65, eligibility response 66, retrieve needed additional eligibility information 68, and additional eligibility information 70, is utilized to calculate patient responsibility 72, and produces a patient financial responsibility sum. A critical step in point-of-service pricing is ensuring the submission of electronic eligibility request directly to the correct health insurer immediately prior to the patient's office visit and receiving a response with patient financial responsibility information, including copay, coinsurance and remaining deductible amounts.

Even with current information on the patient's financial responsibility, to provide accurate point-of-service pricing it must first be determined what the amount a particular health insurer allows for the services provided. This information is provided by each client using the CDM 48 file.

Preferably to calculate a patient payment all point-of-service prices must factor in both the patient's financial responsibility and the allowed amount for a service based on the health plan's fee schedule. The interplay between the various forms of patient financial responsibility and the payer fee schedule in point-of-service pricing calculations is described. There is generally no calculation associated with copay amounts. They are almost always indicated as fixed amounts based upon the terms of the patient's insurance policy, although they may vary based on the type and specialty of the health care provider and where the services were provided: office visit, outpatient facility, and the like.

Preferably estimator engine 10, calculates coinsurance amounts based upon the level of coverage that the patient's insurance policy provides. For example, if the patient's insurance policy indicates that the insurance payer covers 90% of the cost for an office visit, the patient is responsible for the remaining 10%.

A deductible is a specified amount of money that must be paid before the patient's insurance company will pay money towards a claim. To calculate the remaining deductible preferably estimator engine 10, determines how much of the patient's deductible remains unmet in order to calculate an accurate price estimate to present to the patient at the time of service. If the contracted rate is less than the remaining deductible, then this amount is more than likely to be the patient's financial responsibility. On the other hand, if the contracted rate is more than the patient's remaining deductible, then only the actual amount of the remaining deductible becomes the patient's responsibility. Out-of-Pocket Limit, also known as Out-Of-Pocket Maximum is the maximum amount of money you may pay for medical services in a calendar year. Out-of-pocket limit may and may not include deductible depending on insurers' definition of the term. The maximum amount of money spent for health care services also may vary whether they are received in or out-of-network. The dynamic balance adjustment estimator engine 10, of the preferred methodology uses such eligibility information, copays, coinsurance, deductibles and out-of-pocket limits to compute the patient financial responsibility 62.

With reference now to FIG. 4, a typical health insurance plan cost sharing scheme is shown to further illustrate the principals of the present invention. In FIG. 4, in a sample plan, deductible 76, is shown as $1000, with coinsurance 78, being 80%/20%. Insurance company 80, provides an out-of-pocket limit of $6000, and a $20 copayment, for example.

To determine the aforementioned values, preferably, estimator engine 10, first filters by service code 30 (Health Benefit Plan Coverage) and then a search of the Deductible value (EB01=C). This can be in three different categories, depending on each insurance payer: In Network (EB12=Y), Out of Network (EB12=N) or Network Not Specified (EB12=U).

For example, if the EDI 271 returns 2 or more values for the Service Code 30 in the same category (In Network, Out of Network or Network Not Specified), estimator engine 10, would show the minimum Deductible Remaining with its Deductible amount associated.

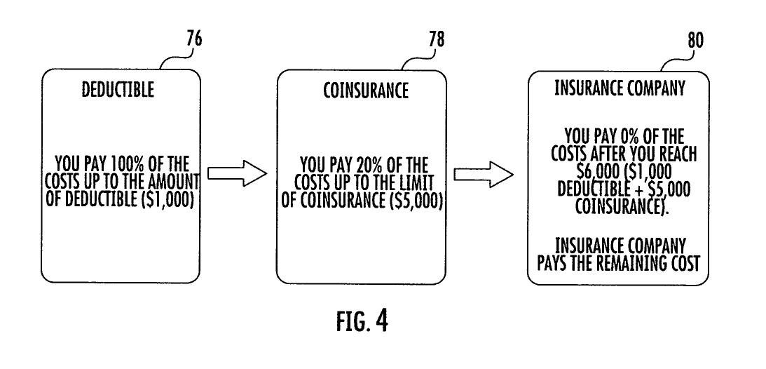

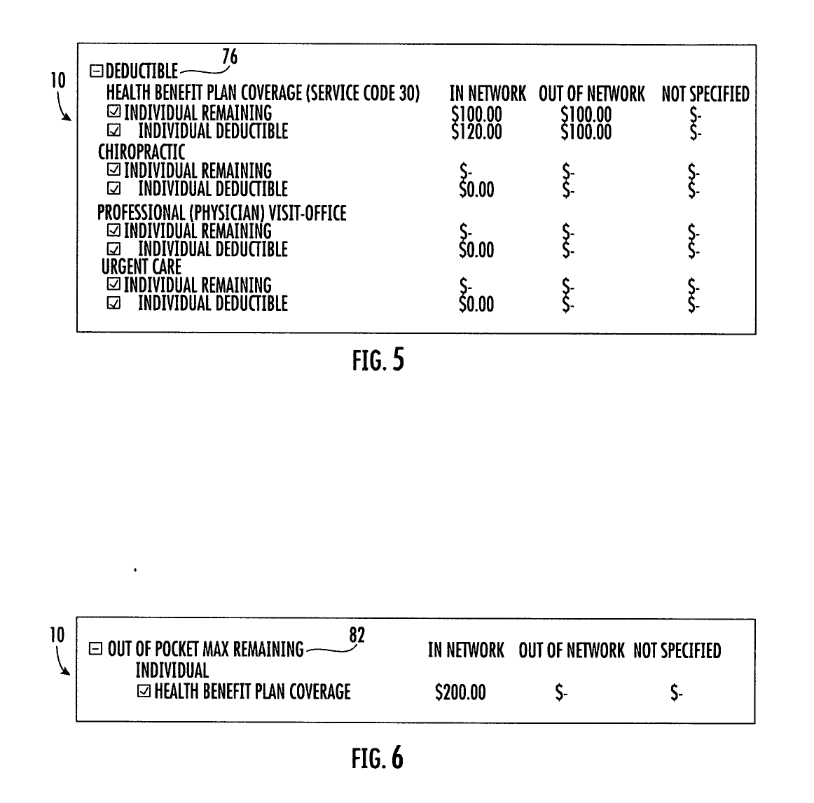

This is seen in FIG. 5, showing an eligibility data view example, where in this example, the Deductible amounts 76, found for the Service Code 30 and category “In Network”, these values and they are preferably shown in the Estimate tab.

In FIG. 6, an example is shown of how the estimator engine 10, of the present invention determines Out of Pocket amounts that the patient will be responsible for. In this example, the Out of Pocket (EB01=G) values 82, from the Payers corresponding category is determined: In Network, Out of Network or Network Not Specified. In the situation that the EDI 271 returns 2 or more values for the same category (In Network—Out Of Network—Not Specified), the minimum Out of Pocket Remaining with its Out of Pocket amount associated would be shown on the screen view.

For the procedure table, this screen is populated with the information related to the selected encounter that a patient may have. Each encounter can have zero, one or multiple charges/CDM/Tiered Pricing (TP)associated to it. Preferably, each patient or client encounter's charge has a procedure id. An important element of this solution is the client's CDM. This is a document that contains all the client's (hospital or practice) charges listed. Each charge has a Current Procedural Terminology (CPT) code, a procedure ID, description, a total or charge amount and a different allowed amount for each payer.

For example:

### Insurance Company A: 40%

### Insurance Company B: 69%

### Insurance Company C: 75%

### Insurance Company D: 70%

Or other insurance provider.

Also, the CDM preferably contains, a Charge Category ID, a Category Name, Department ID and

Department Name.

Accordingly, the procedures table is populated based on the following: Procedure Number: Procedure Number from the Charge or CDM associated to the selected encounter, or the Department Name in case the procedure is TP. Description: Description value of the correspondent CPT in the CDM or TP. Charge Amount: Total Charge value of the correspondent CPT in the CDM or TP. Allowed Amount: Calculation of the Charge Amount multiplied into the percentage covered by the patient's primary insurance. In case of TP, the Allowed Amount is the same as the TP amount. Preferably, the Allowed Amount is always calculated against the patient's primary insurance.

These aforementioned values are preferably retrieved from the EDI 271. A Charge or CDM has a Revenue Code and this Revenue Code has a list of Service Codes associated to it. For example, the Procedure “FACIAL BONES X-RAY” has the Revenue Code “RADIOLOGY DIAGNOSTIC GENERAL” with the Service Codes “Diagnostic X-Ray” and “Health Benefit Plan Coverage”.

In the preferred embodiment, every Service Code has a specific order to search in the EDI 271. In this example, “Diagnostic X-Ray” has order 1 and “Health Benefit Plan Coverage” has order 2. Preferably, there is also a Category order, here, the user would first search in “In Network”, then in “Out of Network” and finally in “Not Specified”.

Preferably, to calculate copay and co-insurance amounts the following methodology is used by estimator engine 10. The values, Copay (EB01=B) and Co-Insurance (EB01=A), are retrieved from the EDI 271 for the private insurances. A charge or CDM has a revenue code and this revenue code has a list of service codes associated to it. In the EDI, the copays and co-insurance values that are specific for the selected CDM record are given. For the Medicare insured patients, preferably the copay values are retrieved from the CDM.

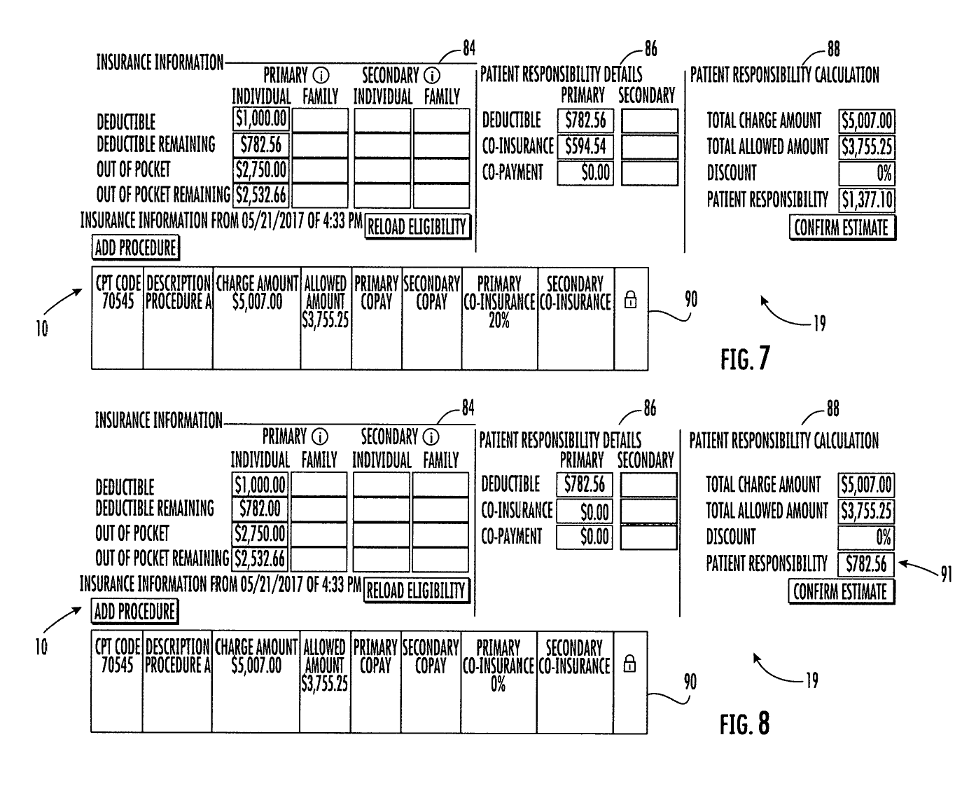

In FIG. 7, shows an example of how estimator engine 10, computes the copay and co-insurance amounts. In FIG. 7, insurance information 84, patient responsibility 86, and patient responsibility calculation 88, add procedure for calculation 90, and display screen 91. Display screen 19, may be any display screen to provide visual output from a computer, tablet, mobile device, or other display mechanism.

### Example 1

Patient Pays Deductible and Co-Insurance. In the following scenario, the patient still hasn't met the deductible. The patient has a $ 782.56 remaining deductible. The procedure's allowed amount is $3,755.25, so the patient will have to pay $782.56 as part of the deductible and the remaining (difference between allowed amount and deductible), $ 3,755.25−$782.56=$2,972.69 has to be multiplied into 0.2 (primary insurance's co-insurance is 20%) to determine the primary insurance co-insurance value=$594.538=$594.54.

So, patient's responsibility is:

$$+ \begin{matrix}\$ 782.56 & ( deductible ) \\ \underline{\$ 592.54 } & ( coinsurance ) \\ \$ 1 , 377.10 & \end{matrix}$$

With reference now to FIG. 8, an example is given of a situation where the patient pays only the deductible, with insurance information 84, patient responsibility 86, and patient responsibility calculation 88, add procedure calculation 90, and display screen 19, shown.

### Example 2

Patient Pays Deductible only:

This example as seen in FIG. 8, differs from that illustrate in FIG. 7, only in the coinsurance value, where instead of being 20%, it's 0%. In this case, the patient only has to pay the deductible amount. Note, that the Deductible should be $3755.25 but the Deductible Remaining is $782.56, that's why the patient, here, pays just $782,56.

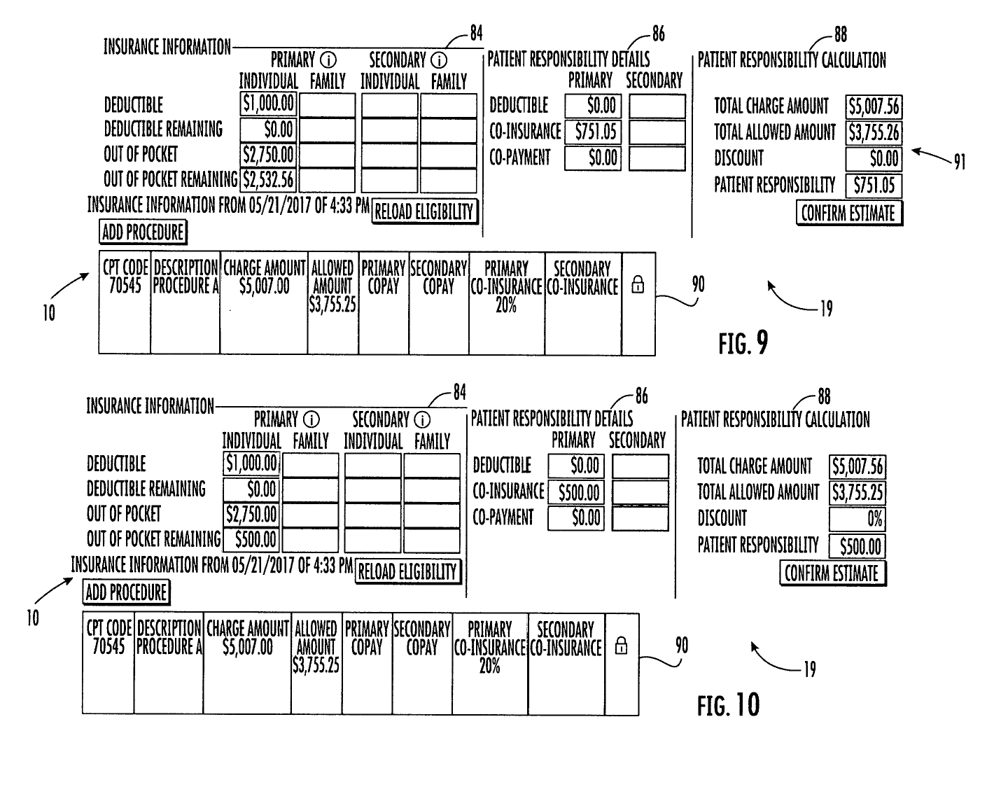

In FIG. 9, an example is shown where the patient pays only the co-insurance amount, with insurance information 84, patient responsibility 86, and patient responsibility calculation 88, add procedure 9, and display screen 19.

### Example 3

Patient pays co-insurance only: in this example, the patient already met the deductible, so only pay the co-insurance 20%), ($3755.25*0.2=$751.05 is due. Note, that the “Out of Pocket Remaining” is higher than $751.05, that is why that amount is shown as Co-Insurance.

With reference to FIG. 10, an example of a situation where a patient pays the deductible and Out of Pocket (OOP) remaining are met. In this example patient information 84, patient responsibility details 86, and the patient responsibility calculation 88, and computer display screen 19, are shown. Here, the patient already met the deductible and only has $500.00 as Out of Pocket remaining, so this means that from the $751.05 the patient would have to pay for the co-insurance ($3755.25*0.2), and only pays $ $500.00, and the remainder is an insurance responsibility.

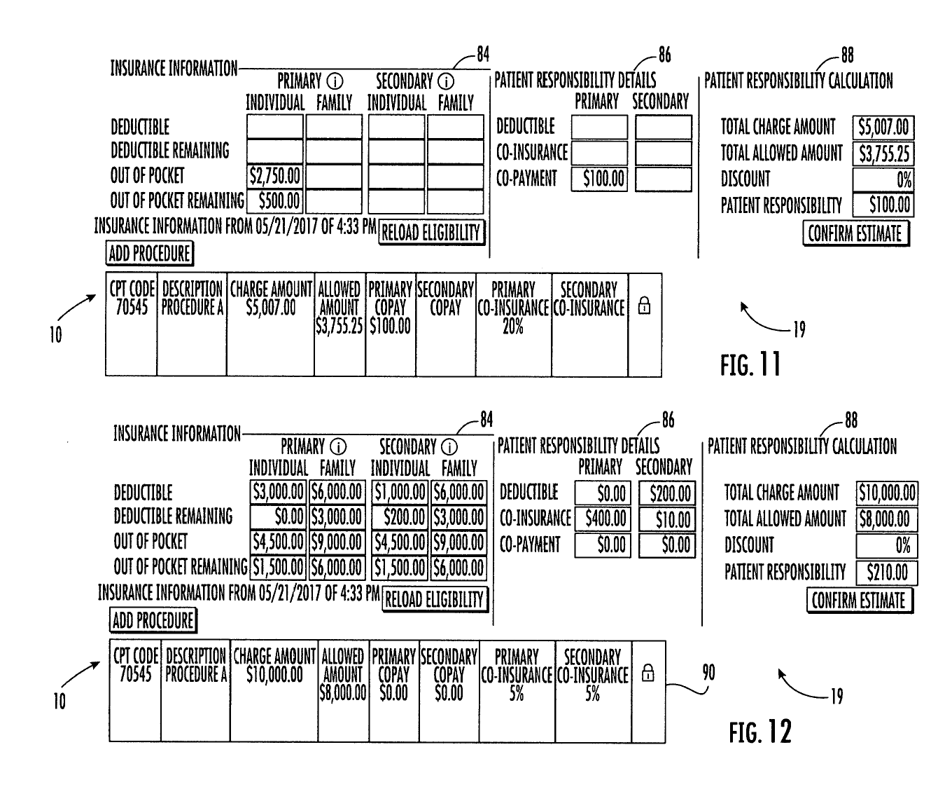

In FIG. 11, an example is illustrated where the information provided is not sufficient to calculate patient responsibility. In this example patient information 84, patient responsibility details 86, and the patient responsibility calculation 88, and display screen 19, are shown. In this example, as no deductible information is present, the same procedure for the out of pocket will apply, here it is not possible to calculate either the deductible nor co-insurance amounts. Only Copay information will be able to be included on the patient responsibility screen.

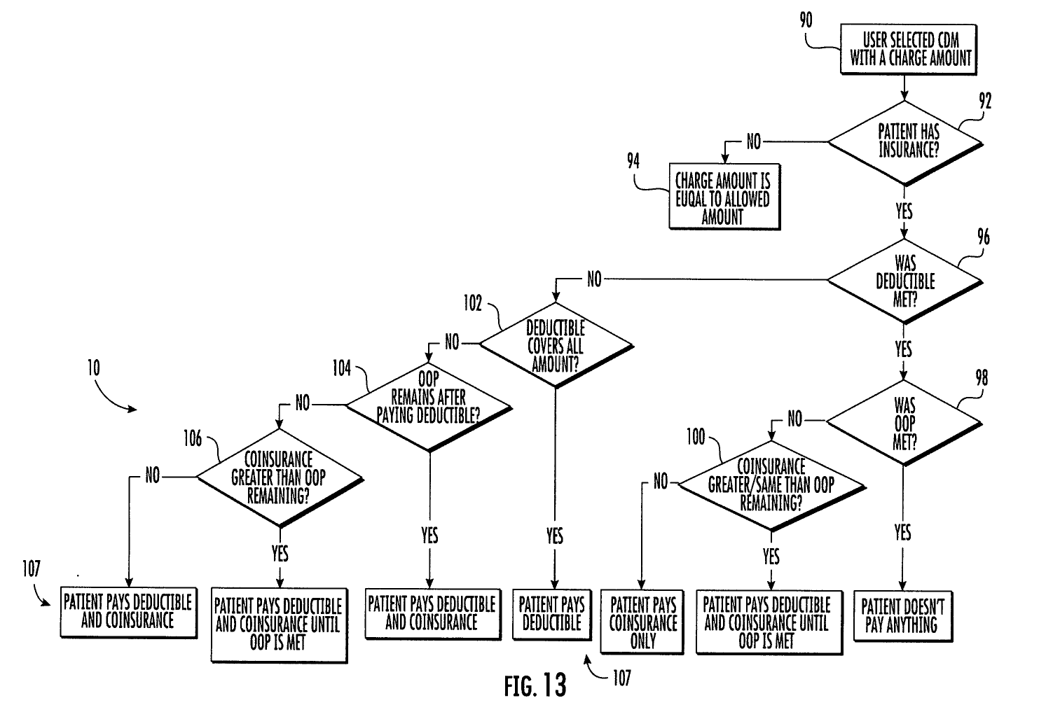

In FIG. 12, an example is shown where a primary insurance deductible was met and secondary information is present for use in estimator engine 10. In this example patient information 84, patient responsibility details 86, and the patient responsibility calculation 88, add procedure 90, and display screen 19, are shown. In this example, the primary insurance information states that the deductible remaining is $0.00 and the secondary insurance information available is that the patient's deductible has not been met yet. Estimator engine 10, preferably uses the patient's remaining deductible and the co-insurance will be calculated not with the difference of the allowed amount and the deductible applied to the user, but instead, in this example, it is calculated based on the secondary insurance. Preferably this is done by multiplying the deductible and the co-payment percentage related to the procedure/s in question. In this following example, the deductible remaining is $200.00 and co-insurance is set to 5% ($10.00) so, the patient will have to end up paying $210.00.

In some situations, providers can choose to integrate their CDM or not into the estimator engine 10. Preferably, the CDM is integrated in estimator engine 10, to be able to produce better and more accurate estimations for the selected procedures. But, in case a provider chooses not to do it, a Tiered Pricing (TP) solution can be used. A set of procedures is provided by estimator engine 10, and is customizable per client, that will allow them to determine a fixed amount to be collected in each case.

Referring now to FIG. 13, a chart illustrates the different business checks used in order to determine the patient payment responsibility in dynamic balance adjustment estimator engine 10. In FIG. 13, user selects CDM with a charge amount 90, then, does patient have insurance 92, and charge amount is equal to allowed amount 94. If patient has insurance 92, then was deductible met 96, if yes, was Out-of Pocket (OOP), met 98, and if yes, then patient does not pay anything. If no, the coinsurance greater/same than OOP remaining 100, if yes, then patient pays the deductible and coinsurance until OOP met. If no, the patient pays coinsurance only.

In FIG. 13, if deductible was met 96, then does deductible cover all charge 102, if yes, then patient pays the deductible. If no, then OOP remaining after paying deductible 104, if yes, then patient pays deductible and coinsurance. If no, then is coinsurance greater than OOP remaining 106, if yes, then patient pays deductible and coinsurance until OOP is met, if no, then patient pays deductible and coinsurance. Patient payment amount is shown in screen 107.

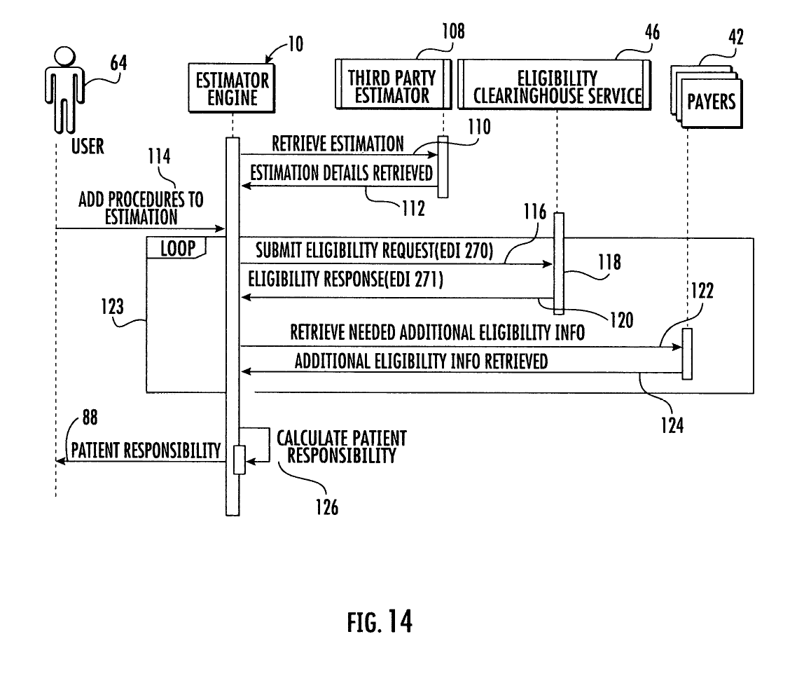

In FIG. 14, shows a preferred method using estimator engine 10, for integrating with existing Estimator systems to retrieve ready-to-use estimates and ability to compare it with dynamic balance adjustment estimator engine 10, calculations. Certain providers may be using an existing estimator system prior to starting using estimator engine 10. Estimator engine 10, seamlessly integrates with multiple third-party estimator systems. Estimator engine 10, preferably compares the patient responsibility retrieved from other system(s) and the one generated in estimator engine 10, and by default selects the highest one.

In FIG. 14, estimator engine 10 is shown with Third party estimator 108, Eligibility Clearing House Service 46, and Payers 42. In a preferred embodiment of the invention, estimator engine 10, retrieves estimation 110, with estimation details received 112. Procedures 114, may be added to the estimation and next, Submit Eligibility request (EDI 270), 116, Eligibility response (EDI 271) 120, and if needed Retrieve needed additional Eligibility information 122, additional eligibility information retrieved 124, loop 123, and then calculate patient responsibility 126, with patient responsibility 88, resulting therefrom.

Another novel feature of estimator engine 10, is that it allows the user to easily identify differences in the calculation of the primary and secondary copay, deductible, coinsurance, total charge amount, total allowed amount and final patient responsibility and to compare the results of estimator engine versus any other estimate generated in an integrated third-party estimator engine. The estimator engine's integrations manager methodology as seen in FIG. 14, not only collects information related to the patient benefits, but also is able to retrieve reports/documents from these systems and make them available to the user, allowing them not to have to switch multiple applications to be able to determine the appropriate patient payment responsibility.

Estimator engine 10, further provides a method of generating estimates for patients existing in the system. Preferably, for patients that already exist in the hospital system, estimator engine 10, has patients demographics and insurance information already retrieved from the hospital system via the various data communication methods explained previously. This information is used to obtain the eligibility information via EDI 270/271 and any missing information is retrieved via the estimator engine integrations manager methodology by communicating directly with the individual payer websites. Based on the eligibility information for the patient's primary and secondary insurance, the patient's responsibility is calculated as previously explained. In addition, estimations already generated in third-party estimator systems being utilized by a hospital are also retrieved and saved within the estimator engine 10, for the staff user to compare and use. Preferably, the estimation once confirmed is assigned to a specific encounter in the estimator engine.

In another preferred embodiment of estimator engine 10, a method of generating estimates for “on-demand” patients calling in with pricing questions. In this application, estimator engine 10, allows a user not only to generate estimations for provider's patients but also, it is possible to perform a price shopping estimation for any self-pay or insured patient that is not a provider's patient yet. By collecting pertinent insurance data and selecting a list of procedures, an estimate can be produced and a reference number will be generated, allowing an estimator engine 10, user to input it into the estimator engine patient estimates section if the price-shopper decided to schedule an appointment after the procedures were estimated using the quick estimate feature of the estimator engine 10.

In operation and use, the present method may be implemented through the operation of a computer, a central data processing unit, wireless communication channels, storage media, computer buses, or other data processing and transfer means well known in the art. Software, cloud computing, internet and other digital means may be utilized, and displayed for example, on the client system 12, such as a hospital billing system, that has computer(s) 19, or other digital processing means, such as tablets, mobile devices, with display screen(s) 17, or other display mechanisms well known in the art. The present invention provides a method and system for an estimator engine for performing dynamic balance estimates and adjustments to reconcile a host hospital or medical provider's patient accounting systems with an automated payment plan management accounting system. The present method may also be used by a hospital billing system vendor within their own system, that is, not as an external system. As such, estimator engine 10, provides an efficient and easy to use methodology for performing dynamic balance adjustments to compute reliable patient payment estimates at or prior to the time of service which enables hospitals or medical providers to increase collections and decrease patient bad-dept. It is also seen that estimator engine 10, further provides a means to characterize what kind of data is being provided for the estimate, account adjustment, interpreting that data, deciding when to apply that data, and then managing the timing of these events. As such, the patient payment estimates and reconciliations timing depends on the type of data and where it is occurring amongst a sequence of other data events.

Estimator engine 10, provides a methodology that can utilize automated interfaces, such as HL7, FTP, CSV, or APA robot, for example, with pre-existing estimating solutions to ensure the contiguous, uninterrupted processing of a patient encounter through a complete workflow, providing patient financial responsibility estimates with great accuracy and ease of operation. The avoidance of duplicate data entry is crucial to ensuring staff can enroll patients in as short as time as possible. The present methodology provides a seamless, fully integrated patient financial responsibility automated payment management solution. The broad library of sophisticated interface technologies allows the present method to connect any pre-existing tools a client has with the patient encounter and financial responsibility estimate workflow, making it seamless and very easy and efficient to use.

Additional advantages and modifications will readily occur to those skilled in the art. The invention in its broader aspects is, therefore, not limited to the specific details, representative apparatus and illustrative examples shown and described. Accordingly, departures from such details may be made without departing from the spirit or scope of the applicant's general inventive concept.

What is claimed is:

1. A dynamic estimator engine for obtaining patient information, encounter information and insurance information from client system, comprising:
receiving patient demographics and visits information via Admission, Discharge, Transfer (ADT) messages in Health Level 7 Protocol (HL7) from the hospital system; and,
reading the incoming HL7 ADT messages, and extracting the needed information from them and use that information to locate the corresponding patient records in the system and update them accordingly.

2. The method of claim 1, further including a method of retrieving advanced eligibility information by integrating with third-party eligibility services and augmenting missing information from individual payer websites using robotics automation.

3. The method of claim 2, wherein an integrations manager connects through a web service (Extensible Markup Language (XML)/Simple Object Access Protocol (SOAP)) with an eligibility clearinghouse service to submit and retrieve healthcare transactions; said clearinghouse services provide synchronous and asynchronous communication methods and both are supported.

4. A method of using eligibility information, charges description manager (CDM), and tiered pricing to compute a patient's payment responsibility, comprising:
calculating the patient payment responsibility utilizing eligibility information, current health plan fee schedule and by each client using said CDM file; and,
determining patient payment calculation including copays, coinsurance, remaining deductible, and out-of-pocket limit is the maximum amount of money you may pay for medical services in a calendar year.

5. The method of claim 4, further including a method of integrating with existing estimator systems to retrieve ready-to-use estimates and ability to compare it with calculated estimates, comprising:
integrating with multiple third-party estimator systems and comparing patient responsibility retrieved from other system(s) and the one generated herein; and,
selecting by default the one with the highest numerical value.

6. The method of claim 5, further including the steps of collecting information related to a patient benefits, and retrieving reports/documents from these systems, thereby allowing their use, and facilitating their application so a user does not to have to switch between multiple applications to be able to determine the appropriate patient payment responsibility.

7. A method using an estimator engine of integrating with existing estimator systems to retrieve ready-to-use estimates and ability to compare it with a calculated estimate, comprising
integrating with multiple third-party estimator systems comparing the patient responsibility retrieved from other system(s) and the one generated said estimator engine and by default selecting the highest one.

8. The method of claim 7, wherein said estimator engine allows a user to easily identify differences in the calculation of the primary and secondary copay, deductible, coinsurance, total charge amount, total allowed amount and final patient responsibility versus any other estimate generated in an integrated third-party estimator engine, thereby allowing a user not to have to switch multiple applications to be able to determine the appropriate patient payment responsibility.

9. The method of claim 7, further including the steps of generating financial estimates for patients existing in the system, comprising:using demographics and insurance information retrieved from the hospital system via the various data communication, utilizing said information to obtain the eligibility information via EDI 270/271, and any missing information is retrieved via said estimator engine by communicating directly with individual payer websites; calculating eligibility information for a patient's primary and secondary insurance.

10. The method of claim 7, wherein estimates already generated in third-party estimator systems being utilized by a hospital are also retrieved and saved within said estimator allowing a user to compare and use.

11. The method of claim 7, further including the steps of generating estimates for “on-demand” patients calling in with pricing questions allowing for the generation of estimations for a provider's patients but also, to perform a price shopping estimation for any self-pay or insured patient that is not a provider's patient yet.

12. The method of claim 7, further including the steps of collecting selected insurance data and selecting a list of procedures, so as to produce an estimate and a reference number.

13. The method of claim 7, wherein said method is implemented through the operation of a computer, or a central data processing unit.

14. The method of claim 7, wherein said method is implemented using wireless communication channels, storage media, computer buses, or other data processing and transfer mechanisms.

15. The method of claim 7, wherein said method utilizes software, cloud computing, internet and other digital operations.
